(19) United States
(12) Patent Application Publication
(10) Pub. No.: US 2023/0169878 A1
(43) Pub. Date: Jun. 1, 2023

(54) COMMUNICATION SYSTEM WITH PRE-CODED SUBROUTINES FOR A VEHICLE

(71) Applicant: GM GLOBAL TECHNOLOGY OPERATIONS LLC, Detroit, MI (US)

(72) Inventors: Matthew E. Gilbert-Eyres, Rochester, MI (US); Russell A. Patenaude, Macomb Township, MI (US); Eric T. Hosey, Royal Oak, MI (US); Matthew Neely, Rochester, MI (US)

(73) Assignee: GM GLOBAL TECHNOLOGY OPERATIONS LLC, Detroit, MI (US)

(21) Appl. No.: 17/539,361

(22) Filed: Dec. 1, 2021

(51) Int. Cl. G09B 5/06; H04N 7/18; A61H 31/00; A61B 5/024; G09B 19/00; A61B 5/00; A61B 5/021; A61M 21/02; H04N 21/414; A61M 21/00

(52) U.S. Cl. CPC G09B 5/065; H04N 7/183; A61H 31/007; A61B 5/02438; G09B 19/003; A61B 5/6802; A61B 5/0031; A61B 5/021; A61M 21/02; H04N 21/41422; A61H 2201/5048; A61M 2021/0027; A61M 2205/3553; A61H 2201/5043

(57) ABSTRACT

A communication system for a vehicle includes an assistance mode for communication between a user of the vehicle and a remote assistance unit. The assistance mode is activable during a concerning situation. The assistance mode includes multiple pre-coded subroutines selectively executable to provide audio instruction and video instruction to the user, via a display in the vehicle. The multiple pre-coded subroutines are triggered by the remote assistance unit. Operating the assistance mode includes relaying of selected data from the vehicle to the remote assistance unit. The selected data includes actions performed by the user. The assistance mode is activable by at least one of the remote assistance unit, the user via the user interface, and the vehicle. The communication system may include a controller adapted to at least partially execute the assistance mode.

### INTRODUCTION

The present disclosure relates generally to a communication system for a vehicle. More specifically, the disclosure relates to a communication system having multiple pre-coded subroutines for communication between a user of the vehicle and a remote assistance unit. It is an undeniable facet of modern life that many people spend a considerable amount of time in their vehicles, while being transported from one place to another. At some point, an occupant of a vehicle may experience a medical situation while inside the vehicle. The occupant may be a driver or a passenger. However, the vehicle may be in a remote location and/or emergency medical services may take some time to arrive. In the interim waiting period, it would be desirable to provide prompt and reliable assistance to the occupant of the vehicle.

### SUMMARY

Disclosed herein is a communication system with an assistance mode for communication between a user of a vehicle and a remote assistance unit. The assistance mode is activable during a concerning situation or event. The assistance mode includes multiple pre-coded subroutines selectively executable to provide audio instruction and video instruction to the user, via a display in the vehicle. The multiple pre-coded subroutines are triggered by the remote assistance unit. Operating the assistance mode includes relaying of selected data to the remote assistance unit. The selected data may include actions performed by the user.

The assistance mode is activable by at least one of the remote assistance unit, the user via the user interface, and the vehicle. In some embodiments, the multiple pre-coded subroutines are stored in the vehicle. In other embodiments, the multiple pre-coded subroutines are stored in a cloud unit interfacing with the controller. The multiple pre-coded subroutines may be updated via remote updates. The communication system may include a controller having a processor and tangible, non-transitory memory on which instructions are recorded, the controller being adapted to at least partially execute the assistance mode.

The audio instruction may include rhythmic beats directing performance of chest compressions when the concerning situation includes a cardiac arrest, with the selected data including a length of time the chest compressions are performed. The multiple pre-coded subroutines may include broadcasting of a video tutorial of a Heimlich maneuver when the concerning situation includes choking. The audio instruction may include broadcasting of calming music when the concerning situation includes a panic attack.

In some embodiments, a camera may be installed in or around the vehicle, with the selected data including real-time imaging data acquired by the camera. The selected data may include sensor data from at least one sensor, including a wearable device worn by the user and/or an electronic sensor implanted in the user. The selected data may include sensor data from at least one sensor installed within the vehicle.

Disclosed herein is a method of operating a communication system for a vehicle having a user interface and a display. The method includes incorporating an assistance mode for communication between a user of the vehicle and a remote assistance unit, the assistance mode being activable during a concerning situation. Multiple pre-coded subroutines may be incorporated in the assistance mode, the multiple pre-coded subroutines being selectively executable to provide audio instruction and video instruction to the user via the display. The method includes relaying of selected data from the vehicle to the remote assistance unit during operation of the assistance mode, the selected data including actions performed by the user.

The method may include obtaining the selected data as sensor data from at least one sensor. The sensor includes a wearable device worn by the user and/or an electronic sensor implanted in the user. The method includes obtaining heart rate and/or blood pressure data from the at least one sensor. The method may include incorporating rhythmic beats in the audio instruction for directing performance of chest compressions when the concerning situation includes a cardiac arrest. The method may include incorporating a length of time the chest compressions are performed into the selected data.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic fragmentary diagram of a communication system for a vehicle;

FIG. 2 is a schematic fragmentary perspective view of a portion of the vehicle of FIG. 1; and

FIG. 3 is a flowchart for a method of operating the communication system of FIG. 1.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

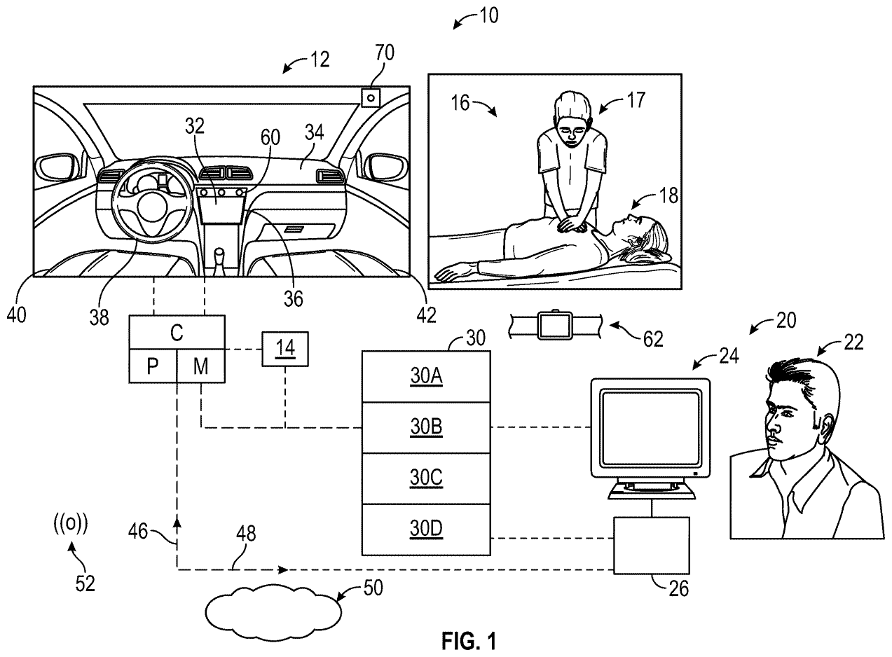

Referring to the drawings, wherein like reference numbers refer to like components, FIG. 1 schematically illustrates a communication system 10 for a vehicle 12. The vehicle 12 may include, but is not limited to, a passenger vehicle, sport utility vehicle, light truck, heavy duty vehicle, minivan, bus, transit vehicle, bicycle, moving robot, farm implement (e.g., tractor), sports-related equipment (e.g., golf cart), boat, plane, train or another moving platform. The vehicle 12 may be an electric vehicle, which may be purely electric or hybrid/partially electric. It is to be understood that the vehicle 12 may take many different forms and have additional components.

The communication system 10 includes an assistance mode 14 for communication between at least one user 16 (e.g., first user 17 and/or second user 18) of the vehicle 12 and a remote assistance unit 20. Referring to FIG. 1, the remote assistance unit 20 includes at least one remote advisor 22 having access to an electronic device 24. The electronic device 24 may be a non-mobile or mobile platform, including but not limited to, a desktop computer, laptop, tablet, cell phone or wearable device. The electronic device 24 includes a control unit 26 having an integrated processor, an integrated memory and other circuitry available to those skilled in the art.

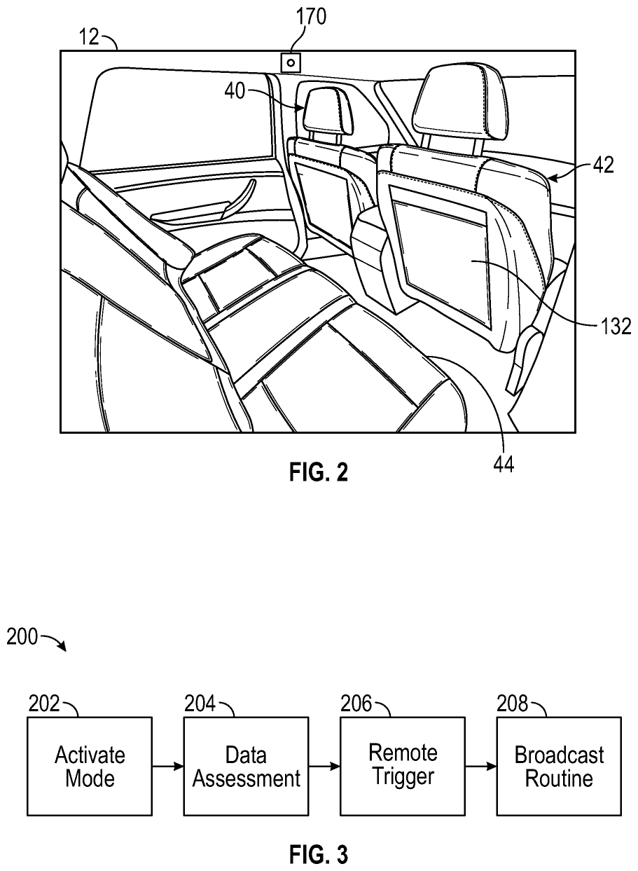

The assistance mode 14 can be activated by the user 16 during a concerning situation. The concerning situation may be a medical event or emergency such as choking, cardiac arrest, panic attack, childbirth etc. The assistance mode 14 may be activated by the remote assistance unit 20 and/or the vehicle 12 automatically upon the occurrence of a predetermined trigger (e.g., deployment of airbags, etc). As described below, the assistance mode 14 includes multiple pre-coded subroutines 30 that are selectively executable to provide audio instruction and video instruction to the user 16 via one or more screens in the vehicle 12, such as display 32 shown in FIG. 1 and/or display 132 shown in FIG. 2.

The positioning of the display 32 may be varied based on the application at hand. For example, referring to FIG. 1, the display 32 may be positioned in a front section of the vehicle 12, such as in the dashboard 34. The display 32 may be integrated in the vehicle radio or infotainment unit 36. Also shown in FIG. 1 are the steering wheel 38, first vehicle seat 40 and second vehicle seat 42. In some embodiments, referring to FIG. 2, the display 132 may be positioned on the posterior portion of the first vehicle seat 40 and/or second vehicle seat 42 to enable viewing by the user 16 when seated in the rear seat 44.

Referring to FIG. 1, a controller C may be employed to facilitate execution of the assistance mode 14. The controller C may be an integral portion of, or a separate module operatively connected to, other controllers of the vehicle 12. Referring to FIG. 1, the controller C has at least one processor P and at least one memory M (or non-transitory, tangible computer readable storage medium) on which instructions are recorded. The memory M can store controller-executable instruction sets, and the processor P can execute the controller-executable instruction sets stored in the memory M.

Referring to FIG. 1, the execution of one of the multiple pre-coded subroutines 30 is triggered by the remote advisor 22 in the remote assistance unit 20. The information flow is bi-directional (schematically indicated by arrows 46, 48 in FIG. 1), with selected data being relayed from the vehicle 12 to the remote assistance unit 20. The selected data includes actions performed by the user 16. Additionally, the communication system 10 provides the ability for remote manipulation of vehicle conditions (e.g., changing the temperature and humidity settings in the vehicle cabin) and components to assist in managing the concerning situation.

In some embodiments, the multiple pre-coded subroutines 30 are stored in the vehicle 12, e.g., in the controller C. In other embodiments, the multiple pre-coded subroutines 30 are stored in a cloud unit 50, shown in FIG. 1. The cloud unit 50 may include one or more servers hosted on the Internet to store, manage, and process data. The cloud unit 50 may be a private or public source of information maintained by an organization, such as for example, a research institute, a company, a university and/or a hospital. The controller C may be configured to communicate with the cloud unit 50 to obtain location coordinates of the vehicle 12 as well as other information. The multiple pre-coded subroutines 30 may updated via remote updates.

Referring to FIG. 1, the communication system 10 may employ a wireless network 52 for transmissions between the remote assistance unit 20 and vehicle 12. The wireless network 52 may be a short-range network or a long-range network. The wireless network 52 may be a communication BUS, which may be in the form of a serial Controller Area Network (CAN-BUS). The wireless network 52 may be a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data. The wireless network 52 may be a Wireless Local Area Network (LAN) which links multiple devices using a wireless distribution method, a Wireless Metropolitan Area Network (MAN) which connects several wireless LANs or a Wireless Wide Area Network (WAN) which covers large areas such as neighboring towns and cities. Other types of network technologies or communication protocols available to those skilled in the art may be employed.

Referring now to FIG. 3, a flowchart of a method 200 of operating the assistance mode 14 is shown. Method 200 need not be applied in the specific order recited herein. Furthermore, it is to be understood that some blocks may be eliminated. In some embodiments, method 200 may be embodied as computer-readable code or stored instructions and may be at least partially executable by the controller C.

Per block 202 of FIG. 3, the assistance mode 14 is activated based on a signal from the user 16. For example, the user 16 may reach out during a concerning situation, via a user interface 60. Referring to FIG. 1, the user interface 60 may be incorporated in the dashboard 34 of the vehicle 12, overhead visor (not shown), or other suitable location. The remote advisor 22 is alerted when the assistance mode 14 is activated and a channel for verbal communication between the user 16 and the remote advisor 22 is initiated.

Proceeding to block 204 of FIG. 3, the method 200 includes an assessment of the situation by the remote advisor 22, based on the verbal communication between the user 16 and the remote advisor 22, as well as selected data. Other types of non-verbal communication, such as typing of messages, may also be employed.

Based on the assessment made in block 204 of FIG. 3, the remote advisor 22 remotely triggers one of the multiple pre-coded subroutines 30, per block 206. The multiple pre-coded subroutines 30 are selectively executable to provide audio instruction and video instruction to the user 16 based on the specifics of the concerning situation and may change vehicle conditions and components to better assist the user 16 in managing the concerning situation. Next, per block 208 of FIG. 3, the selected one of the multiple pre-coded subroutines 30 is broadcast on the display 32 (see FIG. 1) and/or display 132 (see FIG. 2). The remote advisor 22 may simultaneously contact emergency medical services on behalf of the user 16.

In one example, referring to FIG. 1, the multiple pre-coded subroutines 30 include a first subroutine 30A when the concerning situation involves cardiac arrest. For example, if the first user 17 calls in due to a cardiac arrest experienced by the second user 18, the remote advisor 22 may walk the first user 17 through how to give chest compressions. At the same time, the remote advisor 22 may activate the first subroutine 30A in the vehicle 12. This would broadcast a video tutorial on the display 32 (or display 132) while the remote advisor 22 walks through the steps. Once chest compressions start, the remote advisor 22 may activate the chest compression chime sequence that would trigger in vehicle speakers (e.g., in the vehicle's radio or infotainment unit) to produce a rhythmic sound that coordinates with the appropriate chest compression rate. In other words, the audio instruction may include rhythmic beats directing performance of chest compression on the person experiencing the cardiac arrest.

In another example, the multiple pre-coded subroutines 30 may include a second subroutine 30B when the concerning situation involves choking. Here the video instruction may include broadcasting of a tutorial on how to perform a Heimlich maneuver. The multiple pre-coded subroutines 30 may include a third subroutine 30C when the concerning situation involves a panic attack. Here the audio instruction may include broadcasting of calming, soothing music and the video instruction may include a tutorial for guiding someone in the midst of a panic attack. Additionally, the remote advisor may alter or manipulate vehicle conditions, for example, adjusting the temperature in the vehicle 12. The multiple pre-coded subroutines 30 may include a fourth subroutine 30D when the concerning situation involves childbirth. Here the audio instruction may include breathing exercises and the video instruction may include a tutorial with relevant information. Additional subroutines may be added based on the application at hand.

In addition to providing audio and visual effects, the assistance mode 14 may collect and relay selected data from the vehicle 12 to the remote assistance unit 20, including useful information about what actions have been performed. For example, the selected data may include how long the chest compressions have been performed, how long the afflicted user has been experiencing a panic attack, etc.

In some embodiments, referring to FIG. 1, the selected data includes sensor data from a sensor 62. The sensor 62 may be a Bluetooth connected wearable, such as a fitness tracker or smartwatch, worn by the user 16. The sensor 62 may be an electronic sensor implanted in the user 16, such as but not limited to, glucose monitors and cardiac event detectors. The sensor 62 may provide health-related sensor data such as heart rate, pulse oximetry, respiratory rate, and/or blood pressure.

Referring to FIG. 1, the controller C may be adapted to read sensor data from the sensor 62. Additionally, the controller C may be configured to monitor specific changes in the health condition of the user 16 based on the sensor data. For example, a heart attack may be detected when the sensor data indicates high cardiac rhythms. The sensor 62 may be customized for use in a vehicular environment to monitor the health condition of the user 16 while the user 16 is within the vehicle 12. For example, the sensor 62 may be installed on the steering wheel 38, first vehicle seat 40 and/or seat belt (not shown) to detect changes in the health condition of the user 16.

Referring to FIG. 1, the vehicle 12 may include at least one camera installed in or around the vehicle 12, such as first camera 70 shown in FIG. 1 and/or second camera 170 shown in FIG. 2. In some embodiments, the selected data includes real-time imaging data acquired by the first camera 70 and/or second camera 170. The imaging data may be presented in different forms, including but not limited to, captured still images, real-time images and/or digital video signals. “Real-time” as used herein generally refers to the updating of information at the same rate as data is received. More specifically, “real-time” means that the image data is acquired, processed, and transmitted at a high enough data rate and a low enough delay that when the data is displayed, objects move smoothly without user-noticeable judder or latency.

In summary, the communication system 10 (via the assistance mode 14) provides a host of measures to assist a user 16 (e.g., first user 17 and/or second user 18) of the vehicle 12 during a concerning situation. The communication system 10 provides the option for a remote advisor 22 to control vehicle conditions and components to help manage a situation while waiting for first responders to arrive on scene. The assistance mode 14 stores specific pre-coded routines that the remote advisor 22 in the remote assistance unit 20 may select for broadcasting in the vehicle 12.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium with patterns of holes, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a group of files in a file rechargeable energy storage system, an application database in a proprietary format, a relational database energy management system (RDBMS), etc. Each such data store may be included within a computing device employing a computer operating system such as one of those mentioned above and may be accessed via a network in one or more of a variety of manners. A file system may be accessible from a computer operating rechargeable energy storage system and may include files stored in various formats. An RDBMS may employ the Structured Query Language (SQL) in addition to a language for creating, storing, editing, and executing stored procedures, such as the PL/SQL language mentioned above.

The flowcharts illustrate an architecture, functionality, and operation of possible implementations of systems, methods, and computer program products of various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based rechargeable energy storage systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of parameters (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used here indicates at least variations that may arise from ordinary methods of measuring and using such parameters. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A communication system for a vehicle, comprising:
an assistance mode for communication between a user of the vehicle and a remote assistance unit;
wherein the vehicle includes a user interface and a display, the assistance mode being activable during a concerning situation;
wherein the assistance mode includes multiple pre-coded subroutines selectively executable to provide audio instruction and video instruction to the user via the display, the multiple pre-coded subroutines being triggered by the remote assistance unit; and
wherein operating the assistance mode includes relaying of selected data from the vehicle to the remote assistance unit, the selected data including actions performed by the user.

2. The communication system of claim 1, wherein the assistance mode is activable by at least one of the remote assistance unit, the user via the user interface, and the vehicle.

3. The communication system of claim 1, further comprising:
a controller adapted to at least partially execute the assistance mode, the controller having a processor and tangible, non-transitory memory on which instructions are recorded; and
wherein the multiple pre-coded subroutines are stored in the vehicle.

4. The communication system of claim 3, wherein:
the multiple pre-coded subroutines are stored in a cloud unit interfacing with the controller, the multiple pre-coded subroutines being updateable via remote updates.

5. The communication system of claim 1, wherein:
the audio instruction includes rhythmic beats directing performance of chest compressions when the concerning situation includes a cardiac arrest; and
the selected data includes a length of time the chest compressions are performed.

6. The communication system of claim 1, wherein the multiple pre-coded subroutines include broadcasting of a video tutorial of a Heimlich maneuver when the concerning situation includes choking.

7. The communication system of claim 1, wherein the audio instruction includes broadcasting of calming music when the concerning situation includes a panic attack.

8. The communication system of claim 1, further comprising:
a camera installed in or around the vehicle; and
wherein the selected data includes real-time imaging data acquired by the camera.

9. The communication system of claim 1, wherein the selected data includes sensor data from at least one sensor, including a wearable device worn by the user and/or an electronic sensor implanted in the user.

10. The communication system of claim 1, wherein the selected data includes sensor data from at least one sensor installed within the vehicle.

11. A method of operating a communication system for a vehicle having a user interface and a display, the method comprising:
incorporating an assistance mode for communication between a user of the vehicle and a remote assistance unit, the assistance mode being activable during a concerning situation;
incorporating multiple pre-coded subroutines in the assistance mode, the multiple pre-coded subroutines being selectively executable to provide audio instruction and video instruction to the user via the display; and
relaying selected data from the vehicle to the remote assistance unit during operation of the assistance mode, the selected data including actions performed by the user.

12. The method of claim 11, further comprising:
storing the multiple pre-coded subroutines in the vehicle, the multiple pre-coded subroutines being activable by at least one of the remote assistance unit, the user via the user interface, and the vehicle.

13. The method of claim 11, further comprising:
storing the multiple pre-coded subroutines in a cloud unit, the multiple pre-coded subroutines being updateable via remote updates.

14. The method of claim 11, further comprising:
obtaining the selected data as sensor data from at least one sensor; and
wherein the at least one sensor includes a wearable device worn by the user and/or an electronic sensor implanted in the user.

15. The method of claim 14, further comprising:
obtaining heart rate and/or blood pressure data from the at least one sensor.

16. The method of claim 11, further comprising:
incorporating rhythmic beats in the audio instruction for directing performance of chest compressions when the concerning situation includes a cardiac arrest.

17. The method of claim 16, further comprising:
incorporating a length of time the chest compressions are performed into the selected data.

18. The method of claim 11, further comprising:
broadcasting a video tutorial of a Heimlich maneuver in the multiple pre-coded subroutines when the concerning situation includes choking.

19. The method of claim 11, further comprising:
broadcasting calming music in the audio instruction when the concerning situation includes a panic attack.

20. A communication system for a vehicle, comprising:
an assistance mode for communication between a user of the vehicle and a remote assistance unit;
wherein the vehicle includes a user interface and a display, the assistance mode being activable by the user during a concerning situation, via the user interface;
wherein the assistance mode includes multiple pre-coded subroutines selectively executable to provide audio instruction and video instruction to the user via the display, the multiple pre-coded subroutines being triggered by the remote assistance unit;
wherein the audio instruction includes rhythmic beats directing performance of chest compressions when the concerning situation includes a cardiac arrest;
wherein operating the assistance mode includes relaying of selected data from the vehicle to the remote assistance unit, the selected data including a length of time the chest compressions are performed; and
wherein the multiple pre-coded subroutines are stored in a cloud unit and/or the vehicle.
